(19) United States
(12) Patent Application Publication
(10) Pub. No.: US 2026/0091031 A1
(43) Pub. Date: Apr. 2, 2026

(54) CGRP RECEPTOR ANTAGONISTS FOR TREATING CHEMOTHERAPY-INDUCED NAUSEA AND VOMITING (CINV)

(71) Applicant: Alberto CHIARUGI, Firenze (IT)

(72) Inventor: Alberto CHIARUGI, Firenze (IT)

(21) Appl. No.: 19/111,786

(22) PCT Filed: Sep. 13, 2023
(86) PCT No.: PCT/IT2023/050208

§ 371 Date: Mar. 13, 2025

(30) Foreign Application Priority Data
Sep. 14, 2022 (IT) 102022000018768

(51) Int. Cl. A61K 31/496; A61K 31/4545; A61P 1/08

(52) U.S. Cl. CPC A61K 31/496; A61K 31/4545; A61P 1/08

(57) ABSTRACT

A method in which gepants (antagonists of calcitonin gene related peptide (CGRP) receptor) are used for direct treatment of nausea and vomiting including chemotherapy-induced nausea and vomiting (CINV). Among them, drugs frequently prompt nausea and vomiting, such as antitumor chemotherapeutics which often cause CINV which in turn can severely compromise efficacy of therapy, as well as patient quality of life. The method uses antagonists of the CGRP receptor recently approved for migraine treatment to target calcitonin gene related peptide (CGRP), a neuropeptide with pleiotypic effects in the human body to prevent and counteract drug-induced nausea and vomiting including CINV.

### TECHNICAL FIELD

The present invention refers to the field of drugs for the treatment of drug-induced nausea and vomiting

### BACKGROUND ART

It is well known that neoplastic patients often suffer from nausea and vomiting. The latter are very frequently associated with the use of chemicals adopted to counteract cancer growth in these patients. Among the different drugs, antineoplastic chemotherapeutics are those most frequently associated with nausea and vomiting, a condition typically defined “chemotherapy induced nausea and vomiting (CINV). CINV is of remarkable clinical relevance because on the one hand compromises the health status of the patient, and on the other limits dosing and causes treatment interruption.

Molecular mechanisms underlying CINV have been in part understood. To trigger nausea and vomiting drugs act at the level of the brainstem, specifically at the area postrema, also known as “chemoreceptor trigger zone”. Here, neuronal populations capable of detecting different toxic xenobiotics present in blood send projections and activate the vomiting center. These response is of fundamental relevance to survival because allows gastric emptying once a given toxic has been ingested. In light of the potent cytotoxic properties of antitumor chemotherapeutics is makes sense that they promptly activate the chemoreceptor trigger zone and causes CINV. The state of the art, therefore, teaches that mechanisms triggering nausea and vomiting during CINV are different from those involved in nausea and/or vomiting prompted by different conditions such as pregnancy, dizziness, pain or anxiety. Indeed, CINV is prompted by the toxic effects of antitumor chemotherapeutics in the gut as well as to their detection by the chemoreceptor trigger zone, whereas the other forms of nausea and vomiting are not due to activation of the chemoreceptor trigger zone.

Given the high incidence of CINV and its remarkable clinical relevance, the identification of efficacious compounds able to prevent or counteract this disorder has received a great deal of attention. In this regard, the state of the art teaches that antiemetics able to counteract vomiting caused by kinetosis (motion sickness), pregnancy, food intoxication, pain or migraine such as scopolamine, metoclopramide, domperidone, meclizine, doxylamine, dimedrinate or Vitamin B6 are not able to counteract CINV. As mentioned above, this inefficacy is due to the fact that the intrinsic cytotoxic profile of antineoplastic chemotherapeutics detected in the area postrema is primarily responsible for the pathogenesis of CINV. In keeping with the specific mechanisms involved in the pathogenesis of CINV, the latter is counteracted by specific drugs such as antagonists of the serotonin 5HT3 receptors (ondansetron, palosetron, granisoetron), antagonists of neurokinin-1 receptors (aprepitant, netupitant) or corticosteroids (dexamethasone). These compounds, indeed, are not used for the treatment of nausea and vomiting prompted by disorders different from CINV. On this basis, it is not obvious to the expert of the field that antiemetics nonspecific for the treatment of CINV can be used to prevent it. Conversely, the expert of the field knows that the state of the art defines guidelines for the treatment of CINV (New Eng. J. Med., 2016, 374, 1356-67) related to the use of antiemetics specific for the therapy of this disorder.

Unfortunately, it is well known that drugs used for the treatment of CINV are not active in some patients and often tend to lose efficacy during treatment cycles. The technical problem to be solved, therefore, is that of identifying new drugs able to act, alone or in cotreatment paradigms, within the brain regions triggering nausea and vomiting in order to improve therapy of CINV and quality of life of neoplastic patients.

In the field of nausea and vomiting, the underlying neurochemistry is still in part obscure. In this regard, a great deal of attention is focused on the identification of neuropeptides regulating signaling among the different brainstem regions involved in nausea and vomiting. Neuropeptides are proteinaceous, small molecules (10-40 amino acids) able to regulate a myriad of neuronal and endocrine functions. Neuropeptides are released by the classic presynaptic apparatus but, at variance with neurotransmitters such as noradrenaline, acetylcholine or serotonin, generate long lasting signaling that can reach regions distant from the presynaptic terminal (the so called “volume transmission”). Calcitonin gene-related peptide (CGRP) is a 37 amino acid neuropeptide present in the central and peripheral nervous system. The peripheral functions of CGRP are well known and mainly consist in mediating vasodilation and sensitization to pain. In contrast with this, the central functions of CGRP are in large part still unknown. One of the main reasons responsible for this lack of knowledge is the unavailability of brain permeant CGRP receptor agonists and antagonists. Indeed, the current CGRP receptor interacting drugs administered in the periphery are unable to cross the blood brain barrier and regulate CGRP-dependent neurotransmission (Nat. Rev. Neurol., 2018; 14:338). In keeping with this, the expert of the filed knows that to circumvent this pharmacokinetics problem and modulate CGRP neurotransmission the sole strategy is to inject the receptor agonists or antagonists directly into the brain by means of microiontophoresis or intracerebroventricular routes. An additional, recently adopted strategy able to regulate CGRP neurotransmission is the use of viruses that, again directly injected into the brain of experimental animals, carry a genetic information allowing modulation of expression of CGRP or of its cognate receptor. Thanks to these modern gene therapy approaches necessitating direct brain injections, a key role of CGRP in the regulation of neurotransmission among central nervous system regions such as the amygdala, the parabrachial nucleus, the nucleus of the solitary tract, the trigeminal nucleus and several hypothalamic nuclei has recently emerged. However, the expert of the field knows that, just because of the above mentioned impermeability of the blood brain barrier to current CGRP modulating drugs, there is no information in the state of the art regarding the possibility to modulate CGRP neurotransmission in the brain with compounds administered peripherally (i.e, orally, subcutaneously, or intravenously).

A recent advancement of remarkable therapeutic significance is the clinical development of antagonists of the CGRP receptor capable of counteracting the peripheral functions of the neuropeptide. These drugs are named “gepants” and have been approved for the symptomatic and preventive treatment of migraine. The first generation gepants such as olcegepant or telcagepant were efficacious in migraine treatment but caused hepatotoxicity. The second generation gepants such as ubrogepant, atogepant, rimegepant and zavegepant show antimigraine properties but are devoid of any hepatotoxic effect.

The state of the art teaches that gepants do not enter the brain because their structures and polarity render them unable to cross the blood brain barrier (J. Pharmacol. Exp. Ther. 2013; 347:478-86). Accordingly, the state of the art teaches that gepants exert their antimigraine effects by acting at the trigeminovascular afferents within the meninges. Here, gepants counteract the pain sensitizing effects of CGRP and related headache. Thanks to this analgesic effect, gepants also indirectly reduce migraine accompanying symptoms such as phonophobia, photophobia and nausea (Nat. Rev. Neurol. 2018; 14:338-350; Cephalalgia. 2020; 40:924-934; CNS Neurol. Disord. Drug. Targets. 2020; 19:344-359).

Therefore, the expert of the field does not find any teaching in the state of the art that gepants act within the central nervous system and affect neurotransmission. Likewise, the expert of the field does not find any teaching in the state of the art that gepants counteract drug-dependent nausea and vomiting including CINV. This putative effect is not obvious because the state of the art teaches that, in order counteract this type of nausea and vomiting, antiemetics need to reach vomiting centers of the brainstem whereas gepants do not cross the blood brain barrier.

### DISCLOSURE OF THE INVENTION

Unexpectedly, we have now found that the oral (i.e., peripheral) administration of rimegepant, ubrogepant, atogepant and zavegepant causes an accumulation of said gepants within the central nervous system. Further, we have unexpectedly found that the oral administration of rimegepant, ubrogepant, atogepant and zavegepant exerts functional effects in the brain, being able to reduce nausea and vomiting prompted by antitumor chemotherapeutics.

Specifically, we have unexpectedly found that rimegepant, ubrogepant, atogepant and zavegepant are present in brain cortex extracts of rats 6 hrs after the oral administration (3 mg/kg). Animals have been transcardially perfused with cold saline for 10 min in order to remove blood contamination and the possible presence of gepants present in plasma. Upon perfusion, tissue specimens form the cerebral cortex (a region beyond the blood brain barrier) and from the trigeminal ganglion (a region before the blood brain barrier) have been collected and extracted with 10 volumes (weight/volume) of a solution of water/acetonitrile (50%/50%). Tissue extracts have been injected in a LC/MS apparatus to reveal the possible presence of the orally-administered gepants. We have found that gepants are present not only in the trigeminal, but also and unexpectedly in the brain cortex extracts (FIG. 1). Hence, in contrast with the state of the art (J. Pharmacol. Exp. Ther. 2013; 347:478-86), we have unexpectedly found that the gepants rimegepant, ubrogepant, atogepant and zavegepant are able to cross the blood brain barrier and accumulate within the brain parenchyma upon peripheral administration.

We have also unexpectedly found that the oral administration of rimegepant, ubrogepant, atogepant and zavegepant reduces nausea and vomiting in rats exposed to antitumor chemotherapeutics. It is known that the induction of nausea by a chemical can be evaluated in rats measuring repetitive mouth opening (the so called “gaping”) (Autonomic Neuroscience, 2006, 129, 36-41). We have therefore evaluated the effects of rimegepant, ubrogepant, atogepant and zavegepant (3 mg/kg) on gaping prompted in rats by the anticancer drugs cisplatin (6 mg/kg, i.p.) or cyclophosphamide (40 mg/kg, i.p.). These drugs have been injected 6 hrs after the oral administration of the gepants to allow complete tissue drug absorption and distribution. Gaping was monitored for 4 hrs. We have unexpectedly found that the number of gaping events (as an index of nausea) is reduced in animals pretreated with the gepants rimegepant, ubrogepant, atogepant and zavegepant compared to control animals (FIG. 2).

It is known that vomiting can be preclinically induced and evaluated in the shrew (Suncus Murinus). We have unexpectedly found that gepants reduce vomiting in the shrew exposed to anticancer chemotherapeutics. Specifically, we have evaluated the effects of the oral administration of rimegepant, ubrogepant, atogepant and zavegepant (3 mg/kg) on vomiting events induced in the shrew by cisplatin (6 mg/kg i.p.) or cyclophosphamide (40 mg/kg i.p.). To allow complete gepant absorption and tissue distribution, cisplatin and cyclophosphamide have been administered 6 hrs after the gepants and animals monitored for 4 hrs. We have unexpectedly found that vomiting is reduced in animals exposed to rimegepant, ubrogepant, atogepant and zavegepant compared to controls (FIG. 3).

According to the invention, gepants can be formulated and administered via the oral, intravenous, intraarterial, intramuscular, transdermal, endonasal and subcutaneous routes for the treatment of nausea and vomiting. The amounts of gepants to be administered are those commonly adopted for this type of drugs, for instance 10-3000 mg with daily, weekly, or monthly administration.

### BRIEF DESCRIPTION OF DRAWINGS

FIG. 1. Evaluation of the ability of gepants to accumulate within the trigeminal ganglion and cerebral cortex upon oral administration in the rat. Figure shows that 6 hrs after the oral administration of rimegepant, ubrogepant, atogepant and zavegepant (3 mg/kg), the compounds were detectable not only in the trigeminal ganglion but also in the brain cortex extracts of rats (n=5 per group). Animals have been transcardially perfused for 10 min to remove contamination by gepants present in plasma.

FIG. 2. Effects of gepants on nausea induced by cisplatin or cyclophosphamide in the rat. Rats (10 animals/group) received rimegepant, ubrogepant, atogepant and zavegepant orally (3 mg/kg) 6 hrs before an intraperitoneal injection of cisplatin (6 mg/kg) or cyclophosphamide (40 mg/kg). The number of gaping events were evaluated for 4 hrs as an index of nausea. Rats pretreated with rimegepant, ubrogepant, atogepant and zavegepant showed a reduced number of gaping events (and therefore nausea) compared to controls. *p<0.05, **p<0.01 vs Controls, ANOVA and Tukeys post hoc test.

FIG. 3. Effects of gepants on vomiting induced by cisplatin or cyclophosphamide in the shrew. Shrews (10 animals/group) have been orally administered with rimegepant, ubrogepant, atogepant and zavegepant (3 mg/kg) 6 hrs before the intraperitoneal injection of cisplatin (6 mg/kg) or cyclophosphamide (40 mg/kg) and the number of vomiting events evaluated for 4 hrs. Shrews pretreated with rimegepant, ubrogepant, atogepant and zavegepant showed a reduced number of vomiting events compared to controls. *p<0.05, **p<0.01 vs Controls, ANOVA and Tukeys post hoc test.

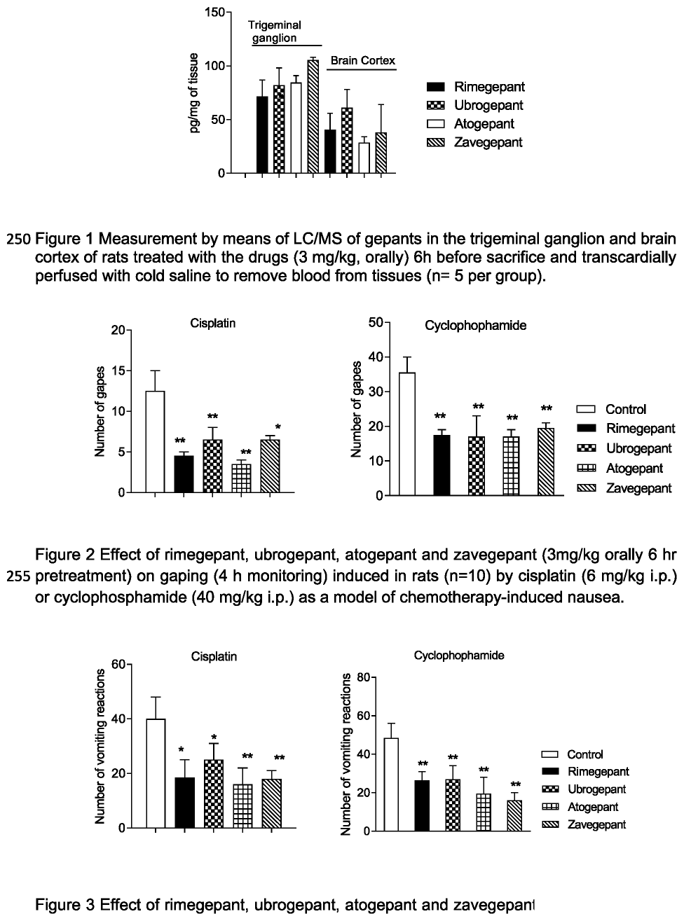

### BEST MODE FOR CARRYING OUT THE INVENTION

The best mode for carrying out the invention is to treat patients before, during and/or after exposure to nausea and/or vomiting inducing agents with daily, weekly or monthly doses of rimegepant, ubrogepant, atogepant and zavegepant administered by different routes such as, for example, but not limited to, oral or intravenous.

1. A method for prevention of chemotherapy-induced nausea and vomiting (CINV) in an individual in need, the method comprising
administering an effective amount of an antagonist of a CGRP receptor or a pharmaceutically acceptable formulation thereof to the individual in need, to the prevent chemotherapy-induced nausea and vomiting (CINV) in the individual.

2. A method for treating chemotherapy-induced nausea and vomiting (CINV) in an individual in need, the method comprising
administering an effective amount of an antagonist of a CGRP receptor and or a pharmaceutically acceptable formulation thereof to the individual in need, to treat chemotherapy-induced nausea and vomiting (CINV) in the individual.

3. A method for prevention or treatment of chemotherapy-induced nausea and vomiting (CINV) in an individual in need, the method comprising administering an effective amount of an antagonist of a CGRP receptor and or a pharmaceutically acceptable formulation thereof to the individual in need, to potentiate the effect of drugs used to prevent or treat chemotherapy-induced nausea and vomiting in the individual.

4.-6. (canceled)

7. The method of claim 1, wherein case said antagonist is Rimegepant.

8. The method of claim 1, wherein said antagonist is Ubrogepant.

9. The method of claim 1, wherein said antagonist is Atogepant.

10. The method of claim 1, wherein said antagonist is Zavegepant.

11. The method of claim 2, wherein said antagonist is Rimegepant.

12. The method of claim 2, wherein said antagonist is Ubrogepant.

13. The method of claim 2, wherein said antagonist is Atogepant.

14. The method of claim 2, wherein said antagonist is Zavegepant.

15. The method of claim 3, wherein said antagonist is Rimegepant.

16. The method of claim 3, wherein said antagonist is Ubrogepant.

17. The method of claim 3, wherein said antagonist is Atogepant.

18. The method of claim 3, wherein said antagonist is Zavegepant.
